(19) United States
(12) Patent Application Publication
(10) Pub. No.: US 2018/0325681 A1
(43) Pub. Date: Nov. 15, 2018

(54) ARTIFICIAL JOINT

(71) Applicant: National Pingtung University Of Science And Technology, Pingtung (TW)

(72) Inventors: Wen-Tzong LEE, Pingtung (TW); Kevin Russell, Jersey City, NJ (US); Raj S. Sodhi, Newark, NJ (US)

(21) Appl. No.: 15/868,047

(22) Filed: Jan. 11, 2018

(30) Foreign Application Priority Data
May 15, 2017 (TW) 106116006

(51) Int. Cl. A61F 2/38

(52) U.S. Cl. CPC A61F 2/384; A61F 2002/30629

(57) ABSTRACT

An artificial joint includes a first engagement structure, a first connecting member, a second engagement structure, and a second connecting member. The first engagement structure has an external gear. The first connecting member is connected to the first engagement structure and configured to connect a femur. The second engagement structure has an internal gear. The external and internal gears are meshed with each other respectively based on a first pitch circle and a second pitch circle. The first pitch circle is greater than the second pitch circle. The center of the second pitch circle is located within the first pitch circle. The second connecting member is connected to the second engagement structure and configured to connect a tibia.

### RELATED APPLICATIONS

This application claims priority to Taiwan Application Serial Number 106116006, filed May 15, 2017, which is herein incorporated by reference.

### BACKGROUND

### Technical Field

The present disclosure relates to an artificial joint, and more particularly, to the artificial joint for knee replacement.

### Description of Related Art

It is now well-understood that human knee motion does not occur in a single plane. Prosthesis designs that restrict knee motion to a single plane (e.g., by implementing a single pin joint or a planar polycentric joint for the knee) will result in the user exhibiting an unnatural gait to compensate for the unnatural prosthetic knee motion.

In recent years, a design method was presented by D'Alessio et al. for a prosthetic knee that replicates the natural spatial motion of the human knee (D'Alessio J., Russell K., Lee W. and Sodhi R. S., “On the Application of RRSS Motion Generation and RRSS Axode Generation for the Design of a Concept Prosthetic Knee,” Mechanics Based Design of Structures and Machines, (in press).). With this method, the fixed and moving axodes for a spatial RRSS (Revolute-Revolute-Spherical-Spherical) linkage that approximates a group of prescribed tibial positions over knee flexion are generated. Because the spatial RRSS axodes for simulating the natural knee motion are noncircular, the engaging structures adopted in the prosthetic knee designed by the foregoing method are also noncircular.

However, there is in theory an indefinite number of possible designs for noncircular engaging structures and no general fabrication method, so in terms of design and manufacturing often face enormous challenges.

### SUMMARY

An aspect of the disclosure is to provide an artificial joint which can replicate natural spatial motions of human knees and be easily designed and manufactured.

According to an embodiment of the disclosure, an artificial joint includes a first engagement structure, a first connecting member, a second engagement structure, and a second connecting member. The first engagement structure has an external gear. The first connecting member is connected to the first engagement structure and configured to connect a femur. The second engagement structure has an internal gear. The external gear and the internal gear are meshed with each other respectively based on a first pitch circle and a second pitch circle. The first pitch circle is greater than the second pitch circle. A center of the second pitch circle is located within the first pitch circle. The second connecting member is connected to the second engagement structure and configured to connect a tibia.

In an embodiment of the disclosure, the second engagement structure is at least a part of a circular gear.

In an embodiment of the disclosure, the artificial joint further includes a shaft and a guiding structure. The shaft is connected to the second engagement structure and passes through the center of the second pitch circle. The guiding structure is connected to the first engagement structure and has at least one arcuate guide groove. The shaft is slidably engaged with the at least one arcuate guide groove.

In an embodiment of the disclosure, the shaft is pivotally connected to the second engagement structure.

In an embodiment of the disclosure, a number of the at least one arcuate guide groove is two. The arcuate guide grooves are symmetrically located at two sides of the second engagement structure. The shaft is extended outwards from the sides of the second engagement structure and slidably engaged between the arcuate guide grooves.

In an embodiment of the disclosure, two ends of the shaft respectively pass through the arcuate guide grooves. The artificial joint further includes two retaining members respectively connected to the ends of the shaft. The guiding structure is retained between the retaining members.

In an embodiment of the disclosure, at least one of the retaining members is detachably connected to the shaft.

In an embodiment of the disclosure, the at least one arcuate guide groove has a centerline. The centerline is at least a part of a circle.

In an embodiment of the disclosure, a center of the first pitch circle coincides with a curvature center of the centerline in a direction parallel to the shaft.

Accordingly, in the artificial joint of the present disclosure, the external gear and the internal gear of the two engagement structures are meshed with each other respectively based on the two pitch circles. That is, the designs and manufacturing of the engagement structures are similar to those of two circular gears. Therefore, the artificial joint of the present disclosure not only can achieve the purpose of replicating natural spatial motions of human knees, but also has the advantages of being easily designed and manufactured.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a perspective view of an artificial joint according to an embodiment of the disclosure;

FIG. 2 is an exploded view of the artificial joint shown in FIG. 1;

FIG. 3 is a schematic diagram illustrating a first pitch circle and a second pitch circle according to an embodiment of the disclosure;

FIG. 4 is a design and manufacturing flow chart of an artificial joint according to an embodiment of the disclosure;

FIG. 5 is a schematic diagram illustrating a tibia moves to different positions relative to a femur;

FIG. 6 is a schematic diagram illustrating a synthesized RRSS (Revolute-Revolute-Spherical-Spherical) linkage according to an embodiment of the disclosure; and

FIG. 7 is a schematic diagram a tibia moves to different positions relative to a femur by using the artificial joint shown in FIG. 1.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

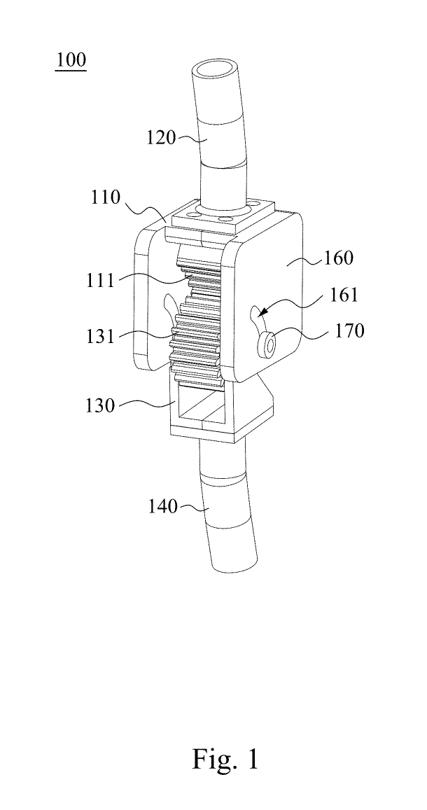

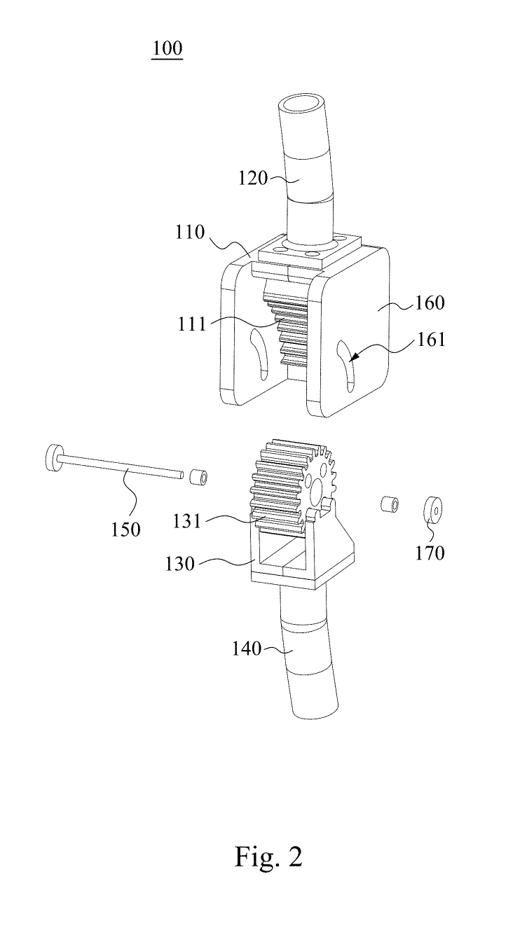

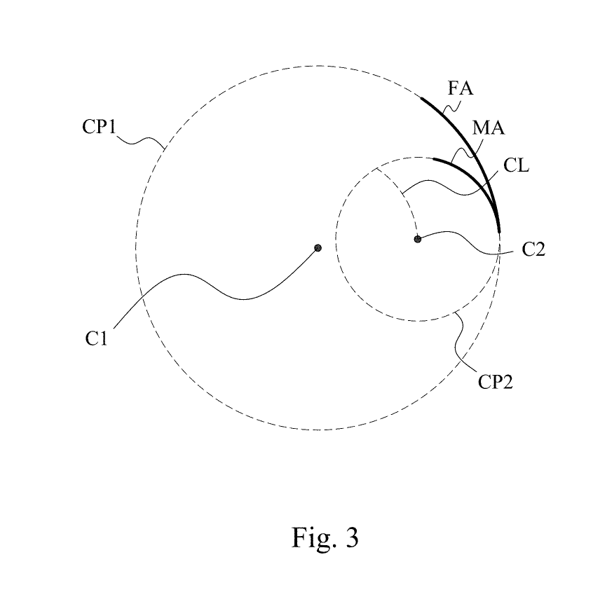

Reference is made to FIGS. 1-3. FIG. 1 is a perspective view of an artificial joint 100 according to an embodiment of the disclosure. FIG. 2 is an exploded view of the artificial joint 100 shown in FIG. 1. FIG. 3 is a schematic diagram illustrating a first pitch circle CP1 and a second pitch circle CP2 according to an embodiment of the disclosure.

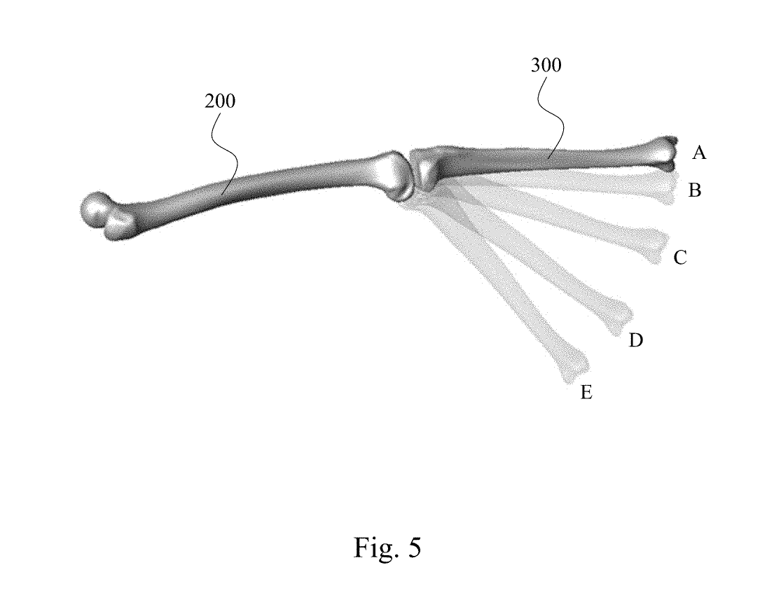

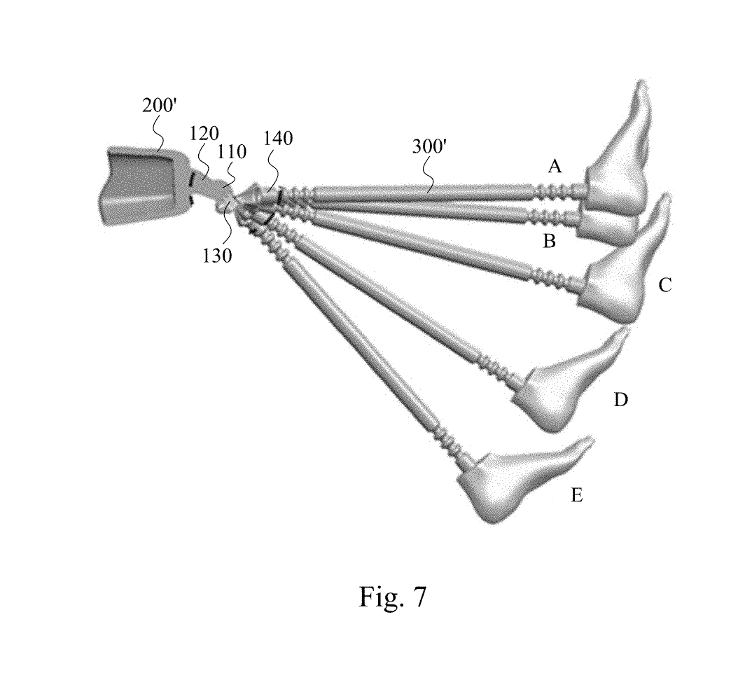

As shown in FIGS. 1-3, in the embodiment, the artificial joint 100 includes a first engagement structure 110, a first connecting member 120, a second engagement structure 130, and a second connecting member 140. The first engagement structure 110 has an external gear 111. The first connecting member 120 is connected to the first engagement structure 110, and is configured to connect a femur 200′ (referring to FIG. 7). For example, the femur 200′ shown in FIG. 7 can be obtained by cutting an end of the femur 200 shown in FIG. 5, and the femur 200′ is then connected to the first connecting member 120. The second engagement structure 130 has an internal gear 131. The external gear 111 and the internal gear 131 are meshed with each other respectively based on the first pitch circle CP1 and the second pitch circle CP2. The first pitch circle CP1 is greater than the second pitch circle CP2. A center C2 of the second pitch circle CP2 is located within the first pitch circle CP1. The second connecting member 140 is connected to the second engagement structure 130, and is configured to connect a tibia 300′ (referring to FIG. 7). For example, the tibia 300′ shown in FIG. 7 is a prosthetic tibia, but the disclosure is not limited in this regard. In some embodiments, the tibia 300′ shown in FIG. 7 can be obtained by cutting an end of the tibia 300 shown in FIG. 5, and the tibia 300′ is then connected to the second connecting member 140.

With the foregoing structural configurations, the second engagement structure 130 can roll on the first engagement structure 110 (by rolling the internal gear 131 on the external gear 111). Hence, the artificial joint 100 of the present embodiment is not a design of prosthetic knee limiting the motions of the tibia 300′ relative to the femur 200′ in a single plane, so as to achieve the purpose of replicating natural spatial motions of human knees.

Furthermore, the external gear 111 and the internal gear 131 meshed with each other respectively based on the first pitch circle CP1 and the second pitch circle CP2 represents that the designs and manufacturing of the first engagement structure 110 and the second engagement structure 130 are similar to those of two circular gears, so that the first engagement structure 110 and the second engagement structure 130 can be easily designed and manufactured than conventional noncircular engagement structures.

In the embodiment, the second engagement structure 130 is at least a part of a circular gear, but the disclosure is not limited in this regard. In some embodiments, the second engagement structure 130 can be a complete circular gear.

In the embodiment, the artificial joint 100 further includes a shaft 150 and a guiding structure 160. The shaft 150 is connected to the second engagement structure 130 and passes through the center C2 of the second pitch circle CP2. That is, the second engagement structure 130 rotates with the shaft 150 as the rotation center. The guiding structure 160 is connected to the first engagement structure 110 and has two arcuate guide grooves 161 (only exemplarily labelling one in each of FIGS. 1 and 2). The arcuate guide grooves 161 are symmetrically located at two sides of the second engagement structure 130. The shaft 150 is extended outwards from the sides of the second engagement structure 130 and slidably engaged between the arcuate guide grooves 161. The arcuate guide grooves 161 are configured to guide the shaft 150, so as to make the external gear 111 and the internal gear 131 be substantially meshed with each other during the second engagement structure 130 rolls relative to the first engagement structure 110.

With reference to FIG. 3, in an embodiment, each of the arcuate guide grooves 161 has a centerline CL. The centerline CL is at least a part of a circle. In addition, a center C1 of the first pitch circle CP1 coincides with a curvature center (overlapped with the center C1 of the first pitch circle CP1 and thus is not labelled) of the centerline CL in a direction parallel to the shaft 150. With the structural configurations, it can be ensured that the external gear 111 and the internal gear 131 are meshed with each other precisely based on the first pitch circle CP1 and the second pitch circle CP2.

In the embodiment, the shaft 150 passes through and is pivotally connected to the second engagement structure 130. Therefore, when assembling, the shaft 150 can sequentially pass through one of the arcuate guide grooves 161 of the guiding structure 160, the second engagement structure 130, and another of the arcuate guide grooves 161, so as to maintain the meshing state between the external gear 111 and the internal gear 131.

Furthermore, in the embodiment, two ends of the shaft 150 respectively pass through the arcuate guide grooves 161. The artificial joint 100 further includes two retaining members 170. The retaining members 170 are respectively connected to the ends of the shaft 150. The retaining members 170 are configured to retain the guiding structure 160 therebetween, so as to prevent the shaft 150 from leaving the arcuate guide grooves 161.

In the embodiment, one of the retaining members 170 is detachably connected to one end of the shaft 150. Therefore, when assembling, said one end of the shaft 150 can pass through the guiding structure 160 and the second engagement structure 130, and then be connected to said one of the retaining members 170 to finish the assembling process. However, the disclosure is not limited in this regard. In practical applications, two of the retaining members 170 can be detachably connected to the shaft 150.

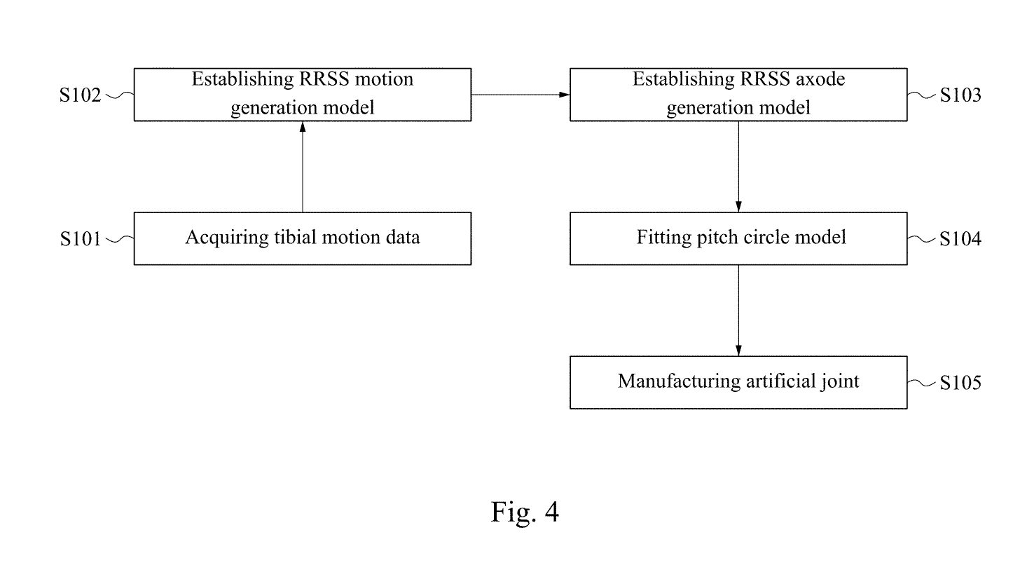

Reference is made to FIG. 4. FIG. 4 is a design and manufacturing flow chart of the artificial joint 100 according to an embodiment of the disclosure. As shown in FIG. 4, the design and manufacturing flow chart of the artificial joint 100 includes steps S101-S105.

In step S101, tibial motion data is acquired. Reference is made to FIG. 5. FIG. 5 is a schematic diagram illustrating the tibia 300 moves to different positions relative to the femur 200. In an embodiment, there are three points p1, q1, and r1 located at an end of the tibia 300 distal to the femur 200, and the tibial motion data includes spatial coordinates of each of the points p1, q1, and r1 respectively corresponding to positions A, B, C, D, and E to which the tibia 300 moves. The spatial coordinates of the points p1, q1, and r1 can be referred to Table 1 shown below. In the embodiment, if the angle of the tibia 300 located at the position A relative to the femur 200 is defined as 0 degree, the angles of the tibia 300 respectively located at the positions B, C, D, and E relative to the femur 200 are 3.25 degrees, 11.03 degrees, 23.86 degrees, and 34.73 degrees.

TABLE 1
Measured coordinates of points p1, q1, and r1 on tibia 300 while
moving to different positions

| Position | Point p1 [mm] | Point q1 [mm] | Point r1 [mm] |
|---|---|---|---|
| A | 40.25, 2.72, −146.97 | 43.73, 2.84, −178.45 | 22.77, −11.56, −186.18 |
| B | 36.42, −6.94, −148.37 | 39.56, −9.38, −179.79 | 19.75, −26.03, −185.84 |
| C | 30.93, −34.02, −148.51 | 33.35, −42.46, −178.94 | 14.96, −61.58, −180.64 |
| D | 25.90, −80.56, −135.79 | 27.55, −97.74, −162.35 | 9.34, −116.53, −157.67 |
| E | 22.38, −117.36, −114.05 | 23.45, −140.56, −135.58 | 4.62, −157.03, −126.60 |

In step S102, a RRSS (Revolute-Revolute-Spherical-Spherical) motion generation model is established.

In step S103, a RRSS axode generation model is established.

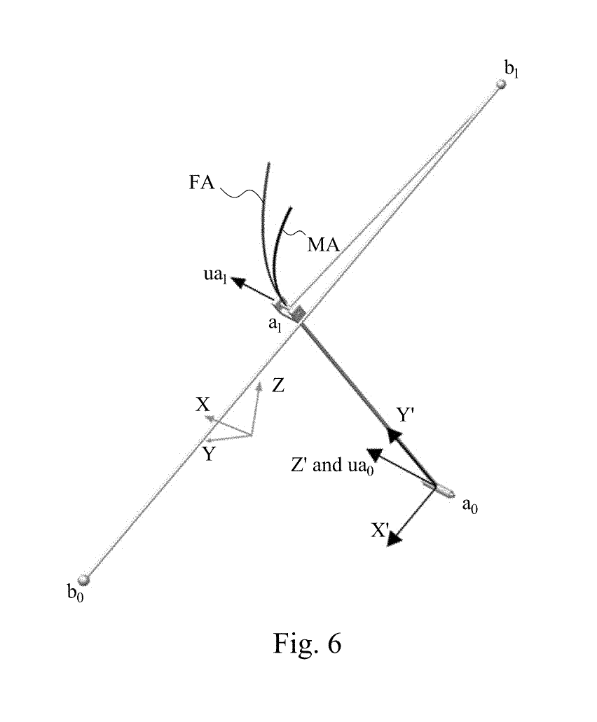

Reference is made to FIG. 6. FIG. 6 is a schematic diagram illustrating a synthesized RRSS linkage according to an embodiment of the disclosure. Initial values and calculated values of the RRSS motion generation model can be referred to Table 2 shown below. Spatial coordinates of each of points p2, q2, and r2 on the synthesized RRSS linkage respectively corresponding to the positions A, B, C, D, and E to which the synthesized RRSS linkage moves can be referred to Table 3 shown below.

TABLE 2
Initial values and calculated values of RRSS motion generation model

| Variables | Initial values | Calculated values |
|---|---|---|
| a0 | 1, −70, −20 | −5.6270, −71.5063, −19.3399 |
| a1 | 1, −23.25, 32 | 8.4156, −12.8436, 29.1038 |
| ua0 | 1, 0, 0 | 0.9826, −0.1655, −0.0844 |
| ua1 | 1, 0, 0 | 0.9826, −0.1655, −0.0844 |
| b0 | 1, 47, −33 | 1.3414, 48.6820, −33.869635 |
| b1 | 1, −65, 86 | 1.51680, −64.2968, 85.9230 |
| θ2~θ5 | 5°, 10°, 15°, 20° | 0.0745°, 0.8106°, 3.7472°, 7.9886° |
| α2~α5 | −5° . . . −5° | −3.7175°, −13.6727°, −31.6497°, |
| | | −48.3064° |

TABLE 3
Coordinates of points p2, q2, and r2 on synthesized RRSS linkage
while moving to different positions

| Position | Point p2 [mm] | Point q2 [mm] | Point r2 [mm] |
|---|---|---|---|
| A | 40.25, 2.72, −146.97 | 43.73, 2.84, −178.45 | 22.77, −11.56, −186.18 |
| B | 38.34, −8.21, −147.85 | 41.49, −10.04, −179.31 | 20.53, −25.00, −185.90 |
| C | 33.71, −36.46, −146.38 | 36.08, −43.19, −177.24 | 15.20, −59.24, −180.81 |
| D | 27.05, −82.95, −132.74 | 28.37, −97.29, −160.94 | 7.81, −114.07, −159.35 |
| E | 22.59, −120.24, −111.46 | 23.28, −140.21, −136.04 | 3.16, −156.56, −130.17 |

The coordinates shown in Table 3 can be perfectly replicated by rolling a moving axode MA onto a fixed axode FA of the synthesized RRSS linkage.

In step S104, a pitch circle model is fitted. It should be pointed out that the RRSS motion generation model is a constrained nonlinear optimization model with 18 unknown dimension variables. Because the solution space for this model has an indefinite number of local minimums and the local minimum calculated is heavily dependent on the initial values specified for each of the model's 18 unknown dimension variables, synthesizing an RRSS motion generator that achieves prescribed positions and also produces circular axodes is extremely challenging.

In an embodiment, to determine the centers and radii of gear pitch circles to replace the noncircular axodes produced by the synthesized RRSS linkage, the method of least squares was employed in this work. The method of least squares satisfies the following Equation (1):

$$F ( h , k , r ) = {\sum }_{i = 1}^{N} {[ {( {x}_{i} - h )}^{2} + {( {y}_{i} - k )}^{2} - {r}^{2} ]}^{2} \qquad ( 1 )$$

In which (xᵢ, yᵢ) represent points of the fixed axode FA and the moving axode MA of the synthesized RRSS linkage on plane X′-Y′ shown in FIG. 6, r represents a radius of a pitch circle, and (h, k) represents a coordinate of a center of the pitch circle. With the method of least squares, “best fit” means Equation (1) is minimized. Therefore, the first pitch circle CP1 and the second pitch circle CP2 shown in FIG. 3 can be fitted.

In step S105, the artificial joint 100 is manufactured. After the first pitch circle CP1 and the second pitch circle CP2 are calculated, the first engagement structure 110 having the external gear 111 and the second engagement structure 130 having the internal gear 131 can be easily manufactured in a manner similar to the case of manufacturing circular gears.

Reference is made to FIG. 7. FIG. 7 is a schematic diagram the tibia 300′ moves to different positions relative to the femur 200′ by using the artificial joint 100 shown in FIG. 1. There are three points p3, q3, and r3 located at an end of the tibia 300′ distal to the femur 200′, and spatial coordinates of each of the points p3, q3, and r3 respectively corresponding to the positions A, B, C, D, and E to which the tibia 300′ can be referred to Table 4 shown below.

TABLE 4
Coordinates of points p3, q3, and r3 on tibia 300′ while moving to
different positions

| Position | Point p3 [mm] | Point q3 [mm] | Point r3 [mm] |
|---|---|---|---|
| A | 40.25, 2.72, −146.97 | 43.73, 2.84, −178.45 | 22.77, −11.56, −186.18 |
| B | 37.79, −8.59, −147.90 | 40.86, −10.48, −179.36 | 20.00, −25.63, −185.84 |
| C | 32.48, −36.40, −146.44 | 34.70, −43.13, −177.31 | 14.19, −59.71, −180.64 |
| D | 25.89, −82.73, −132.87 | 27.11, −97.07, −161.08 | 7.48, −114.85, −158.87 |
| E | 22.72, −120.27, −111.07 | 23.54, −140.37, −135.53 | 4.90, −157.99, −128.56 |

By comparing the data in Table 1 and the data in Table 4, it can be clearly seen that the scalar differences between the coordinates of the points p3, q3, and r3 on the tibia 300′ shown in FIG. 7 and the coordinates of the points p1, q1, and r1 shown in FIG. 5 are very small. Therefore, it is obvious that the artificial joint 100 designed by the design and manufacturing flow chart of FIG. 4 certainly can achieve the purpose of replicating natural spatial motions of human knees, and the concept of circular gears can certainly be applied to manufacturing prosthetic knees based on RRSS axodes.

It should be pointed out that the artificial joint of present disclosure is not limited to be manufactured by using the design and manufacturing flow chart of FIG. 4.

According to the foregoing recitations of the embodiments of the disclosure, it can be seen that in the artificial joint of the present disclosure, the external gear and the internal gear of the two engagement structures are meshed with each other respectively based on the two pitch circles. That is, the designs and manufacturing of the engagement structures are similar to those of two circular gears. Therefore, the artificial joint of the present disclosure not only can achieve the purpose of replicating natural spatial motions of human knees, but also has the advantages of being easily designed and manufactured.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

1-9. (canceled)

10. A method of designing and manufacturing an artificial joint, comprising:
acquiring tibial motion data;
establishing a RRSS (Revolute-Revolute-Spherical-Spherical) motion generation model according to the tibial motion data;
establishing a RRSS axode generation model according to the RRSS motion generation model;
fitting a pitch circle model according to the RRSS axode generation model; and
manufacturing the artificial joint according to the pitch circle model.

11. The method of claim 10, wherein there are a plurality of points located at an end of a tibia distal to a femur, and the tibial motion data comprises spatial coordinates of each of the points on respectively corresponding to a plurality of positions to which the tibia moves.

12. The method of claim 10, wherein the RRSS axode generation model comprises a fixed axode and a moving axode, and the pitch circle model comprises a first pitch circle and a second pitch circle respectively fitted by the fixed axode and the moving axode.

13. The method of claim 12, wherein the fitting comprises:
fitting the first pitch circle and the second pitch circle respectively from the fixed axode and the moving axode by using the method of least squares.

14. The method of claim 12, wherein the artificial joint comprises a first engagement structure and a second engagement structure, and the manufacturing comprises:
manufacturing an external gear of the first engagement structure according to the first pitch circle;
manufacturing an internal gear of the second engagement structure according to the second pitch circle; and
making the external gear and the internal gear be meshed with each other respectively based on the first pitch circle and the second pitch circle.

15. The method of claim 14, further comprising:
connecting a shaft to the second engagement structure and passing the shaft through a center of the second pitch circle;
connecting a guiding structure to the first engagement structure, wherein the guiding structure has at least one arcuate guide groove; and
making the shaft be slidably engaged between the at least one arcuate guide groove, so as to make the external gear and the internal gear be meshed with each other during the second engagement structure rolls relative to the first engagement structure.
